(19) United States
(12) Patent Application Publication
(10) Pub. No.: US 2022/0262455 A1
(43) Pub. Date: Aug. 18, 2022

(54) DETERMINING THE GOODNESS OF A BIOLOGICAL VECTOR SPACE

(71) Applicant: Recursion Pharmaceuticals, Inc., Salt Lake City, UT (US)

(72) Inventors: Berton EARNSHAW, Cedar Hills, UT (US); James JENSEN, Centerville, UT (US); Renat KHALIULLIN, Salt Lake City, UT (US); Nathan LAZAR, Salt Lake City, UT (US)

(73) Assignee: Recursion Pharmaceuticals, Inc., Salt Lake City, UT (US)

(21) Appl. No.: 17/179,043

(22) Filed: Feb. 18, 2021

(51) Int. Cl. G16B 5/20; G06N 3/04; G06N 3/08; G16B 40/00

(52) U.S. Cl. CPC G16B 5/20; G16B 40/00; G06N 3/08; G06N 3/0454

(57) ABSTRACT

A system for determining a goodness of a deep learning model comprises a memory coupled with a processor. The processor accesses a first set of vectors representative of images of a biological assay. The vectors of the first set of vectors are outputs of a first deep learning model. The processor creates a first distribution of a first plurality of pairwise comparisons of vectors, of the first set of vectors, which were generated from image pairs with similar cell perturbations. The processor creates a second distribution of a second plurality of pairwise comparisons of vectors, of the first set of vectors, which were generated from image pairs with dissimilar cell perturbations. The processor determines a difference between the first distribution and the second distribution and uses the difference to make a determination of goodness of the deep learning model as applied to the biological assay.

### BACKGROUND

Industrialized drug discovery can involve a continuous, iterative loop of “biology and bits” where wet lab biology experiments are executed automatically. For example, in an experimental assay, disease states may be induced in one or more cell types and then automatically screened alongside healthy cells using specific fluorescent probes. By applying potential drug compounds to the diseased cells, signals of experimental efficacy can be identified, “rescue” of diseased cells to a healthy state can be identified, and signals of potential side-effects can be identified. An assay may be conducted on a microplate with hundreds or over a thousand wells, in which these cell/drug interactions are tested. In one assay, many of these microplates may be run as a batch (e.g., at the same time or sequentially over a very short period such as on the same day); or in multiple-batches that are run at different times (e.g., batches may be separated by hours, days, or weeks). Consequently, a voluminous amount of data is generated.

To handle the large amount of data, automation is utilized. Images of the cells in an assay are captured, and machine learning models (e.g., deep learning models) then transform the images of the cells in a tested assay into a list of numbers called vectors. The vectors are intended to represent the biology of the image within the vectors of a vector space, hopefully without representing any of the nuisance or confounding information in the image. Once a collection of images from an assay are transformed (such as by a neural network) into vectors, the vectors naturally become members of a mathematical set called a vector space. The vectors in this vector space can then be analyzed using analytical techniques, which may be embodied in and automated by software, to determine results of an assay or results of a combination of assays. It should be appreciated that for different ways of turning images into vectors (e.g., using different models), the arrangement of data as vectors within the vector space can be very different with each model. In some cases, a model may be used to transform images from two or more assays into vectors.

### BRIEF DESCRIPTION OF DRAWINGS

The accompanying drawings, which are incorporated in and form a part of the Description of Embodiments, illustrate various embodiments of the subject matter and, together with the Description of Embodiments, serve to explain principles of the subject matter discussed below. Unless specifically noted, the drawings referred to in this Brief Description of Drawings should be understood as not being drawn to scale. Herein, like items are labeled with like item numbers.

FIG. 1 shows a block diagram of a system for determining a goodness of a deep learning model, in accordance with various embodiments.

FIG. 2 illustrates a first distribution which represents similarities between a first plurality of pairwise comparisons of vectors of a vector space and a second distribution which represents similarities between a second plurality of pairwise comparisons of vectors of the vector space, in accordance with various embodiments.

FIG. 3 illustrates a first distribution which represents similarities between a first plurality of pairwise comparisons of vectors of a vector space and a second distribution which represents similarities between a second plurality of pairwise comparisons of vectors of the vector space, in accordance with various embodiments.

FIG. 4 illustrates a first distribution which represents similarities between a first plurality of pairwise comparisons of vectors of a vector space and a second distribution which represents similarities between a second plurality of pairwise comparisons of vectors of the vector space, in accordance with various embodiments.

FIG. 5 illustrates components of an example computer system, with which or upon which, various embodiments may be implemented.

FIGS. 6A-6E illustrate a flow diagram of determining a goodness of a deep learning model, in accordance with various embodiments.

### DESCRIPTION OF EMBODIMENTS

Reference will now be made in detail to various embodiments of the subject matter, examples of which are illustrated in the accompanying drawings. While various embodiments are discussed herein, it will be understood that they are not intended to limit to these embodiments. On the contrary, the presented embodiments are intended to cover alternatives, modifications and equivalents, which may be included within the spirit and scope the various embodiments as defined by the appended claims. Furthermore, in this Description of Embodiments, numerous specific details are set forth in order to provide a thorough understanding of embodiments of the present subject matter. However, embodiments may be practiced without these specific details. In other instances, well known methods, procedures, components, and circuits have not been described in detail as not to unnecessarily obscure aspects of the described embodiments.

### Overview of Discussion

A mathematical space that represents features of the biology of an image of cells in an assay as mathematical vectors is called a “vector space.” A vector space may also be interchangeably called an a “biological vector space,” an “image space,” or a “feature space.” When images represent similar cell biology, it is desirable to represent similar outcomes in their respective vectors of a vector space so as to demonstrate consistency. However, it is undesirable for the consistency in the vectors to be so strong that vectors fail to preserve and represent relevant differences (diversity) in cell biology. Accordingly, there is a balance to be struck between preserving consistency while also preserving relevant diversity between biology in different images in the representative vectors of a vector space.

Along these lines, one question that can arise after a vector space is created is “How good are the vectors in this vector space at representing relevant biology, or features, of the cells in the images.” Another question that may arise after several different experiments are run is, “How good are vectors from images of one experiment as compared to vectors from images of another experiment at representing the relevant biology of cells in transformed images.” Yet another question that may arise is: “How good is a model at maintaining consistency and/or preserving diversity in relevant biology of cells of images when the images are collected from different assays in the same or different batches of an experiment.” Additional questions may arise regarding the amount of noise or non-relevant biology which is encoded, by a deep learning model, from images of cell biology into vectors of a vector space, especially as compared to another deep learning model. As will be described herein, answers to these and other questions can be articulated through the use of metrics which allow goodness of vectors of a vector space (or the model which was used to create the vectors) to benchmark metrics, threshold metrics, and/or metrices from other biological feature spaces. Herein, processes for creating some metrics for describing a goodness of a vector space, and the vectors therein, and allowing for comparisons or evaluations of its relative goodness are described along with several example applications for use of these metrics.

With a sensitive metric that is properly calibrated to discern the differences between relevant biology encoded from an image into vectors of a vector space, choices can be made between the many alternatives that a deep learning model approach to vectorizing cell biology of images may offer. For example, models may be selected based on their goodness of maintaining both consistency and diversity (as compared to other models). This facilitates having much more faithful readouts that are much more relatable across many plates in an experiment. Similarly, models may be selected which better maintain consistency and diversity (as compared to other models) across many experiments that are separated in time. This allows the time-separated vectors in a vector space to be aggregated in a manner that facilitates making higher confidence decisions from the combined datasets rather than making individual decisions scoped only to individual experiments or portions thereof.

Discussion begins with a description of notation and nomenclature. Discussion then shifts to description of an example system for determining a goodness of a deep learning model. Techniques for generating distributions from vectors representative of images of a biological assay are described. Metrics for measuring the difference between two such distributions are then described, where the difference is a measure of the separation between a pair of distributions. Some examples of distributions and measures of difference between them are depicted and described. Some components of an example computer system are then described. Finally, an example method for determining a goodness of a deep learning model is then described, with reference to the system, computer system, and illustrated examples.

### Notation and Nomenclature

Some portions of the detailed descriptions which follow are presented in terms of procedures, logic blocks, processes, modules and other symbolic representations of operations on data bits within a computer memory. These descriptions and representations are the means used by those skilled in the data processing arts to most effectively convey the substance of their work to others skilled in the art. In the present application, a procedure, logic block, process, module, or the like, is conceived to be one or more self-consistent procedures or instructions leading to a desired result. The procedures are those requiring physical manipulations of physical quantities. Usually, although not necessarily, these quantities take the form of electrical or magnetic signals capable of being stored, transferred, combined, compared, and otherwise manipulated in an electronic device/component.

It should be borne in mind, however, that all of these and similar terms are to be associated with the appropriate physical quantities and are merely convenient labels applied to these quantities. Unless specifically stated otherwise as apparent from the following discussions, it is appreciated that throughout the description of embodiments, discussions utilizing terms such as “accessing,” “creating,” “determining,” “using,” “comparing,” “selecting,” “adjusting,” “comparing,” “performing,” “providing,” “displaying,” “storing,”or the like, refer to the actions and processes of an electronic device or component such as: a processor, a memory, a computer system or component(s) thereof, or the like, or a combination thereof. The electronic device/component manipulates and transforms data represented as physical (electronic and/or magnetic) quantities within the registers and memories into other data similarly represented as physical quantities within memories or registers or other such information storage, transmission, processing, or display components.

Embodiments described herein may be discussed in the general context of computer/processor executable instructions residing on some form of non-transitory computer/processor readable storage medium, such as program modules or logic, executed by one or more computers, processors, or other devices. Generally, program modules include routines, programs, objects, components, data structures, etc., that perform particular tasks or implement particular abstract data types. The functionality of the program modules may be combined or distributed as desired in various embodiments.

In the figures, a single block may be described as performing a function or functions; however, in actual practice, the function or functions performed by that block may be performed in a single component or across multiple components, and/or may be performed using hardware, using software, or using a combination of hardware and software. To clearly illustrate this interchangeability of hardware and software, various illustrative components, blocks, modules, circuits, and steps have been described generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example hardware described herein may include components other than those shown, including well-known components.

The techniques described herein may be implemented in hardware, or a combination of hardware with firmware and/or software, unless specifically described as being implemented in a specific manner. Any features described as modules or components may also be implemented together in an integrated logic device or separately as discrete but interoperable logic devices. If implemented in software, the techniques may be realized at least in part by a non-transitory computer/processor readable storage medium comprising computer/processor readable instructions that, when executed, cause a processor and/or other components of a computer or electronic device to perform one or more of the methods described herein. The non-transitory computer/processor readable data storage medium may form part of a computer program product, which may include packaging materials.

The non-transitory computer readable storage medium (also referred to as a non-transitory processor readable storage medium) may comprise random access memory (RAM) such as synchronous dynamic random access memory (SDRAM), read only memory (ROM), non-volatile random access memory (NVRAM), electrically erasable programmable read-only memory (EEPROM), FLASH memory, compact discs, digital versatile discs, optical storage media, magnetic storage media, hard disk drives, other known storage media, and the like. The techniques additionally, or alternatively, may be realized at least in part by a computer readable communication medium that carries or communicates code in the form of instructions or data structures and that can be accessed, read, and/or executed by a computer or other processor.

The various illustrative logical blocks, modules, circuits and instructions described in connection with the embodiments disclosed herein may be executed by one or more processors, such as host processor(s) or core(s) thereof, digital signal processors (DSPs), general purpose microprocessors, application specific integrated circuits (ASICs), application specific instruction set processors (ASIPs), field programmable gate arrays (FPGAs), graphics processing unit (GPU), microcontrollers, or other equivalent integrated or discrete logic circuitry. The term “processor” as used herein may refer to any of the foregoing structures or any other structure suitable for implementation of the techniques described herein. In addition, in some aspects, the functionality described herein may be provided within dedicated software modules or hardware modules configured as described herein. Also, the techniques, or aspects thereof, may be fully implemented in one or more circuits or logic elements. A general purpose processor may be a microprocessor, but in the alternative, the processor may be any conventional processor, controller, microcontroller, or state machine. A processor may also be implemented as a combination of computing devices, e.g., a plurality of microprocessors, one or more microprocessors in conjunction with an ASIC or DSP, or any other such configuration or suitable combination of processors.

### Example System for Determining the Goodness of a Deep Learning Model

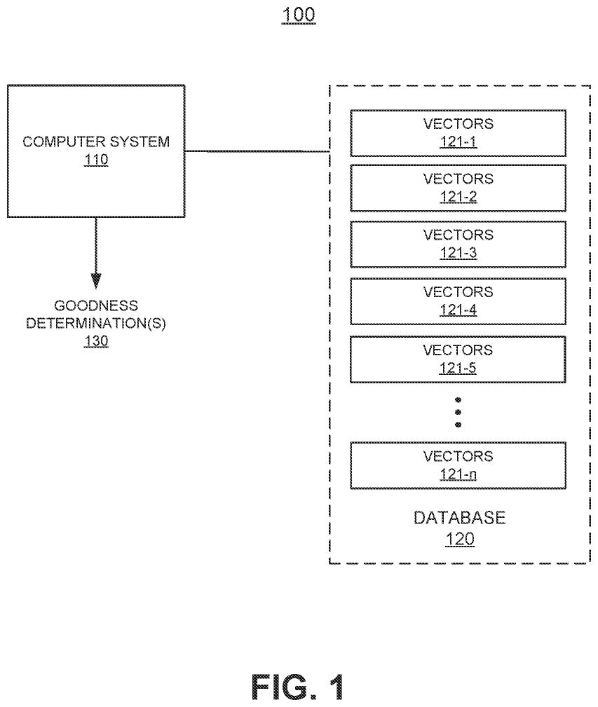

FIG. 1 shows a block diagram of a system 100 for determining the goodness of a deep learning model, in accordance with various embodiments. System 100 includes a computer system 110. In some embodiments, system 100 may include or else access one or more stores of vectors 121, such as database 120.

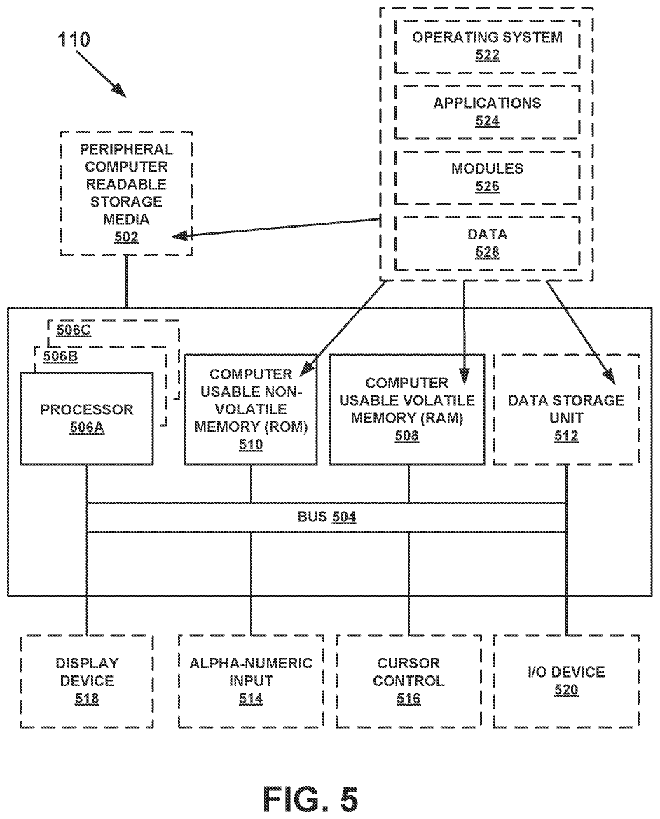

Computer system 110, as will be described in more detail in FIG. 5, includes at least a processor and a memory. The computer system 110 operates to access a one or more sets of vectors created by one or more models (e.g., one or more deep learning models) from images of the biology of cells, create distributions from the set of vectors, measure a difference between the distributions, and then use the difference to make a determination of goodness. The determination may be with respect to a single model; goodness of a vector space which is populated with vectors generated by a model; and/or with respect to a comparison of models. This determination can take many forms and may be provided as an output 130 from the computer system 110. As will be discussed, in some techniques this difference may be measured as the separation or distance, which may be referred to as the “spread,” between two distributions.

A database 120, or other storage, includes one or more sets of vectors 121 (e.g., 121-1, 121-2, 121-3, 121-4, 121-5 . . . 121-n). Each set of vectors 121 (e.g., 121-1) comprises vectors which are representative of the internal biology, state of the cells, and the morphology of the of the population of the cells within each of the images of cells of a biological assay that has been transformed by a model (e.g., a deep learning model or other model or technique) into a vector of the particular set of vectors. It should be appreciated that, in some embodiments, one or more databases or stores of sets of vectors 121 may be included in computer system 110. Each set of vectors resides in a vector space. Depending on how many dimensions are represented by a set of vectors 121, it may occupy the same vector space or a different vector space than another set of vectors 121.

As discussed previously, biological assays often take place in numerous wells on a microplate (where numerous may be hundreds or a thousand or more wells), each with cells and each with a particular perturbation (which may be no perturbation, such as for control). For purposes of ease of explanation, and not of limitation, a basic assay which has two types of perturbations to cells will be described. In this basic assay, cells from the same cell line are placed in numerous test wells of a microplate and then perturbed in one of two ways (e.g., such as being left alone or being treated with a drug candidate). The assay may take place on a single microplate, on two or more microplates that are run simultaneously or sequentially in a single experiment, or in separate experiments that are time-separated (e.g., accomplished hours, days, or weeks or more apart). Images of the biology of cells in these wells, after being converted to vectors of a vector space, can be analyzed in a number of different ways. However, such analysis is not the subject this disclosure; instead, this disclosure concerns determining a goodness of the vector space which has been accessed.

By way of example, and not of limitation: set of vectors 121-1 consists of vectors which are transformed by a first model (e.g., a first deep learning model) from images of test wells across microplates in a first experiment conducted at a first time. Set of vectors 121-2 consists of vectors which are transformed by a second model (e.g., a second deep learning model that is different from the first deep learning model) from images of test wells across microplates in a second experiment conducted at a second time that is separate and distinct from the first time. Set of vectors 121-3 consists of vectors which are transformed by the first model from images of test wells across microplates in a second experiment conducted at a second time that is separate and distinct from the first time (e.g., two months later). Vectors 121-3 can be compared with vectors 121-1 to check for consistency or to vectors 121-2 to benchmark the first model against the second model. Set of vectors 121-4 consists of vectors which are transformed by the second model from images of test wells across microplates in the first experiment conducted at the first time. Vectors 121-4 can be compared to vectors 121-1 to benchmark the first model against the second model. Set of Vectors 121-5 consists of vectors which are transformed by the first model from images of test wells of a first microplate in the first experiment. Set of vectors 121-n consists of vectors which are transformed by the first model from images of test wells of a second microplate in the first experiment (where the first microplate and the second microplate are different microplates).

Although wells and microplates in an experiment may be treated in the same way and may include cells from the same cell line and use the same perturbations, differences in outcome represented in cell biology of a well can occur based on one or some combination of factors. For example, wells located near the center of a microplate may be exposed to slightly different experimental conditions than well on an edge region; wells on the first microplate in a 100 microplate experiment may experience less evaporation than wells in the 100th microplate of the experiment; similarly wells in different experiments or different batches of the same experiment may experience slightly different experimental conditions (e.g., differences in conditions such as temperature, humidity, concentration of a perturbant, age of a cell line, etc.). Some of these differences may be expressed as unspecified noise in the vectors of the vector space. Different models used to transform images into vectors may encode different amounts of noise.

With continued reference to FIG. 1, in various embodiments, computer system 110 accesses or otherwise receives vectors (e.g., vectors 121-1) representative of images of a biological assay, where vectors 121-1 are an output of a first deep learning model (as has been described).

Computer system 110, or a portion thereof such as a processor, creates a first distribution which represents similarities between subset of vectors (e.g., a subset of vectors 121-1) generated from image pairs with similar cell perturbations. The first distribution is a cumulative distribution function (CDF) created by cumulating similarities that are measured in a selected manner between vectorizations of pairs of images which have the same biology (e.g., same cell line perturbed in the same way). There are many ways of measuring similarity. For example, any suitable distance measurement may be used, with smaller distance differences representing greater similarity between an evaluated pair than larger distance differences. One example way to measure similarity is to measure the difference in cosine between like vectors that are associated with different images of a pair being evaluated. In this example, the cosines value for an evaluated pair will vary between 0 and 1, with a value closer to zero representing greater similarly and a value closer to 1 representing less similarity. Another example way to measure similarity is to measure the Euclidian distance (also referred to as the L2) distance between like vectors that are associated with different images of a pair being evaluated. In this example, a smaller Euclidian distance represents greater similarly and a larger Euclidian distance represents less similarity.

Computer system 110, or a portion thereof such as a processor, also creates a second distribution which represents similarities between a second plurality of pairwise comparisons of vectors of the set of vectors (e.g., vectors 121-1). Vectors in each of the pairwise comparisons are generated from image pairs with dissimilar cell perturbations. The second distribution is a cumulative distribution function (CDF) created by cumulating similarities that are measured in a selected manner between vectorizations of pairs of images which have the dissimilar biology (e.g., same cell line perturbed in a first way for one of the images of a pair and in a second, different way for the second image of the pair). In various embodiments, similarity of the evaluated pairs in the second distribution is measured in the same manner as was selected for measuring similarity between evaluated pairs in the first distribution.

Computer system 110, or a portion thereof such as a processor, determines a difference between the first distribution and the second distribution. In some embodiments, as depicted, the difference may be the “spread,” which may be a measure of separation between the first and second distributions. In some embodiments, the difference may be determined by a parametric test that makes some assumptions about the distributions. In some embodiments, the measure of the difference is non-parametric and may be the outcome of a statistical test. One example of a non-parametric measurement of the differences is obtained by performing a Kolmogorov-Shapiro test (also known as a “K-S test”) on the first and second distributions to find the K-S test statistic for the two distributions (e.g., the largest vertical distance (i.e., “spread”) between the two distributions). In another example, the average separation across the distributions may be determined and used as the difference. In yet another example, the Wilcoxon Rank Sum test may be performed on the first and second distributions and its resulting test statistic may be determined and used as the difference between the two distributions. In some embodiments, a parametric test of difference, which makes similar assumptions about the data being compared, can be used to determine a difference. One example of a parametric test is the T-test which can be used to compare the means of two or more groups (i.e., distributions) of data. Other parametric and/or non-parametric techniques may be used to measure a difference between two distributions.

Computer system 110, or a portion thereof such as a processor, can then use the difference as a metric to make a determination 130 of goodness. The goodness determination 130 may be output, such as to a printer or display; stored; and/or provided to a designated location/entity. Generally, the larger the difference the better the underlying model was at being both consistent and preserving diversity when transforming the cell biology of the images into vectors of the vector space. The determination may be made by comparing the difference to a benchmark or threshold and then judging the relative goodness by whether the difference is less than, the same as, or greater than the benchmark. In other embodiments, differences calculated similarly for different sets or subsets of vectors can also be compared to determine a relative goodness in comparison to one another (with the larger difference of the two being better or having a greater goodness). In this manner, differences generated and compared for sets or subsets of vectors can be compared to determine how well a model transforms the cell biology of images into vectors or how well different models compare at transforming cell biology of images into vectors. By way of example and not of limitation, some uses of the difference metric include: comparing two sets of vectors created for different experiments (separated in time) using the same model in order to measure goodness of the model across experiments; comparing two sets of vectors created for the same experiment (e.g., the same images) but with different models in order to measure the relative goodness of the different models to one another; and comparing two sets of vectors created for different microplates within the same experiment using the same model in order to measure a goodness of the model across the experiment (encoding of noise may be detected if the goodness changes beyond a permissible threshold).

### Example Distributions and Difference Metrics

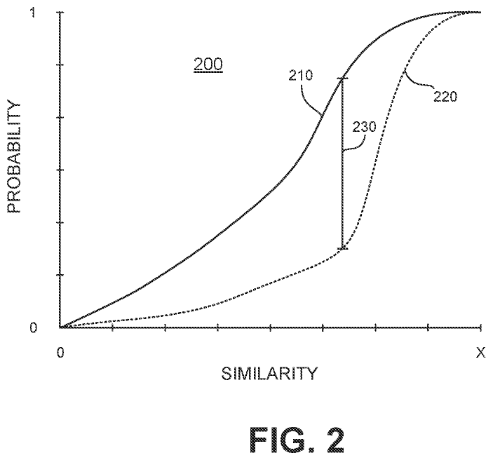

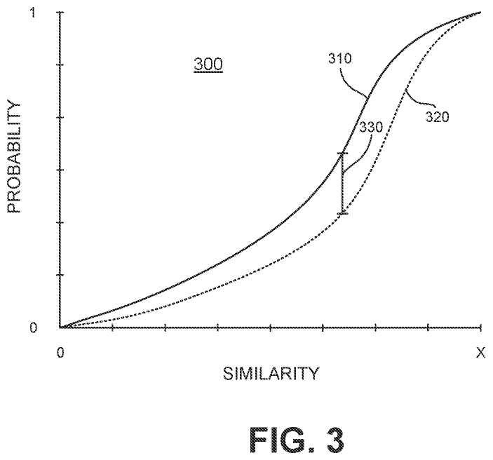

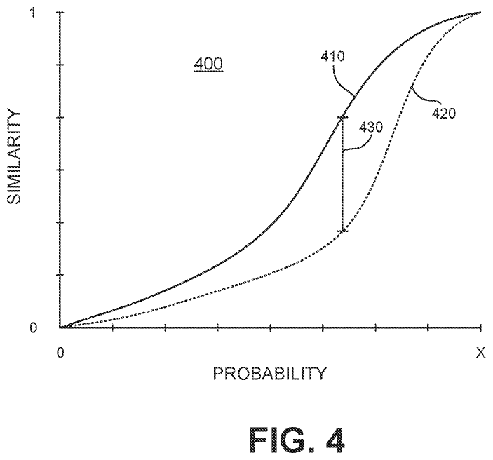

Several examples of distributions and differences are shown in FIG. 2, FIG. 3, and FIG. 4. Although these figures depict graphs, computer system 110 may or may not output such a graph (such as on a display). For purposes of example, the pairwise comparisons in all three Figures are created in the same way (e.g., measuring the cosine of the angle between evaluated vectors of a pairwise comparison) and then creating a distribution of the cosine measurements for each type of pairwise comparisons (e.g., one distribution for same/same pairwise comparisons and one distribution for same/different pairwise comparisons). Additionally, the differences in all three figures are determined in the same way (e.g., by calculating a K-S test statistic of the largest vertical separation between the two compared distributions). This sameness across the three figures in the manner of creating the distributions and determining the difference between them facilities comparison of the difference metrics to one another in each of the three figures. It should be appreciated that other techniques and tests described herein may be similarly employed in creation of distributions and comparisons of the distributions.

FIG. 2 illustrates a graph 200 showing a first distribution 210 which represents similarities between a first plurality of pairwise comparisons of a subset of vectors (e.g., a subset of vectors 121-1 and a second distribution which represents similarities between a second subset of vectors 121-1, in accordance with various embodiments. As previously described, vectors 121-1 is a set of vectors transformed by a first model (e.g., a first deep learning model) from images of test wells across microplates in a first experiment conducted at a first time. First distribution 210 is the result of same/same pairwise comparisons of vectors transformed from images in which the pairs being evaluated have similar biology (e.g., the same cell line and the same perturbations). Second distribution 220 is the result of same/different pairwise comparisons of vectors transformed from images in which the pairs being evaluated have dissimilar biology (e.g., the same cell line but different perturbations). Difference 230 illustrates the largest vertical separation between distributions 210 and 220. Other parametric and/or non-parametric techniques may be used to measure a difference between two distributions.

FIG. 3 illustrates a graph 300 showing a first distribution 310 which represents similarities between a first plurality of pairwise comparisons of a subset of set of vectors 121-4 and a second distribution which represents similarities between a second plurality of pairwise comparisons of a second subset of the set of vectors 121-4, in accordance with various embodiments. As previously described, vectors 121-4 is a set of vectors which are transformed by the second model from images of test wells across microplates in the first experiment conducted at the first time. First distribution 310 is the result of same/same pairwise comparisons of vectors transformed from images in which the pairs being evaluated have similar biology (e.g., the same cell line and the same perturbations). Second distribution 320 is the result of same/different pairwise comparisons of vectors transformed from images in which the pairs being evaluated have dissimilar biology (e.g., the same cell line but different perturbations). Difference 330 illustrates the largest vertical separation between distributions 310 and 320. Other parametric and/or non-parametric techniques may be used to measure a difference between two distributions.

FIG. 4 illustrates a graph 400 showing a first distribution 410 which represents similarities between a first plurality of pairwise comparisons of a subset of set of vectors 121-3 and a second distribution 420 which represents similarities between a second plurality of pairwise comparisons of a second subset of set of vectors 121-3, in accordance with various embodiments. As previously described, vectors 121-3 is a set of vectors transformed by the first model from images of test wells across microplates in a second experiment conducted at a second time that is separate and distinct from the first time (e.g., two months later). First distribution 410 is the result of same/same pairwise comparisons of vectors transformed from images in which the pairs being evaluated have similar biology (e.g., the same cell line and the same perturbations). Second distribution 420 is the result of same/different pairwise comparisons of vectors transformed from images in which the pairs being evaluated have dissimilar biology (e.g., the same cell line but different perturbations). Difference 430 illustrates the largest vertical separation between distributions 410 and 420. Other parametric and/or non-parametric techniques may be used to measure a difference between two distributions.

The large separation illustrated by difference metric 230 indicates the first deep learning model is preserving both consistency of similar relevant biology and diversity of dissimilar relevant biology. When difference metric 230 is compared to difference metric 330 (which has a large drop in comparative magnitude), it is evident that the first deep learning model used for creating vectors 121-1 has a higher relative goodness than the second deep learning model used to create vectors 121-4. When difference metric 230 is compared to difference metric 430 (which has slightly less magnitude), it is evident that the first deep learning model used for creating both vectors 121-1 and vectors 121-3 has a strong relative goodness across experiments, which is a sign that it does not encode a large amount of non-relevant information (e.g., noise from whatever the source).

### Example Computer System Environment

FIG. 5 illustrates components of an example computer system 110, with which or upon which, various embodiments may be implemented. With reference now to FIG. 5, all or portions of some embodiments described herein are composed of computer-readable and computer-executable instructions that reside, for example, in computer readable storage media of or accessible by a computer system. It is appreciated that computer system 110 of FIGS. 1 and 5 is only an example and that embodiments as described herein can operate on or within a number of different computer systems including, but not limited to, general purpose networked computer systems, embedded computer systems, routers, switches, server devices, client devices, various intermediate devices/nodes, stand-alone computer systems, media centers, handheld computer systems, multi-media devices, and the like.

System 110 includes an address/data bus 504 for communicating information, and a processor 506A coupled with bus 504 for processing information and instructions. As depicted in FIG. 5, system 110 is also well suited to a multi-processor environment in which a plurality of processors 506A, 506B, and 506C are present. Conversely, system 110 is also well suited to having a single processor such as, for example, processor 506A. Processors 506A, 506B, and 506C may be any of various types of microprocessors. Computer system 110 also includes data storage features such as a computer usable volatile memory 508, e.g., random access memory (RAM), coupled with bus 504 for storing information and instructions for processors 506A, 506B, and 506C. System 110 also includes computer usable non-volatile memory 510, e.g., read only memory (ROM), coupled with bus 504 for storing static information and instructions for processors 506A, 506B, and 506C.

In some embodiments a data storage unit 512 (e.g., a magnetic or optical disk and disk drive) is coupled with bus 504 for storing information and instructions.

In some embodiments, computer system 110 is well adapted to having peripheral computer readable storage media 502 such as, for example, a floppy disk, a compact disc, digital versatile disc, other disc based storage, universal serial bus flash drive, removable memory card, and the like coupled thereto.

Computer system 110 may also include an optional alphanumeric input device 514 including alphanumeric and function keys coupled with bus 504 for communicating information and command selections to processor 506A or processors 506A, 506B, and 506C. Computer system 110 may also include an optional cursor control device 516 coupled with bus 504 for communicating user input information and command selections to processor 506A or processors 506A, 506B, and 506C. In some embodiments, system 110 also includes an optional display device 518 coupled with bus 504 for displaying information.

Optional cursor control device 516 allows the computer user to dynamically signal the movement of a visible symbol (cursor) on a display screen of display device 518 and indicate user selections of selectable items displayed on display device 518. Alternatively, it will be appreciated that a cursor can be directed and/or activated via input from optional alphanumeric input device 514 using special keys and key sequence commands. Computer system 110 is also well suited to having a cursor directed by other means such as, for example, voice commands.

In some embodiments, computer system 110 also includes an I/O device 520 for coupling system 110 with external entities. For example, in one embodiment, I/O device 520 is a modem for enabling wired or wireless communications between system 110 and an external device or network such as, but not limited to, the Internet.

Referring still to FIG. 5, various other components are depicted for system 110. Specifically, when present, an operating system 522, applications 524, modules 526, and data 528 are shown as typically residing in one or some combination of computer usable volatile memory 508 (e.g., RAM), computer usable non-volatile memory 510 (e.g., ROM), and data storage unit 512. In some embodiments, all or portions of various embodiments described herein are stored, for example, as an application 524 and/or module 526 in memory locations within RAM 508, computer readable storage media within data storage unit 512, peripheral computer readable storage media 502, and/or other computer readable storage media.

### Example Methods of Operation

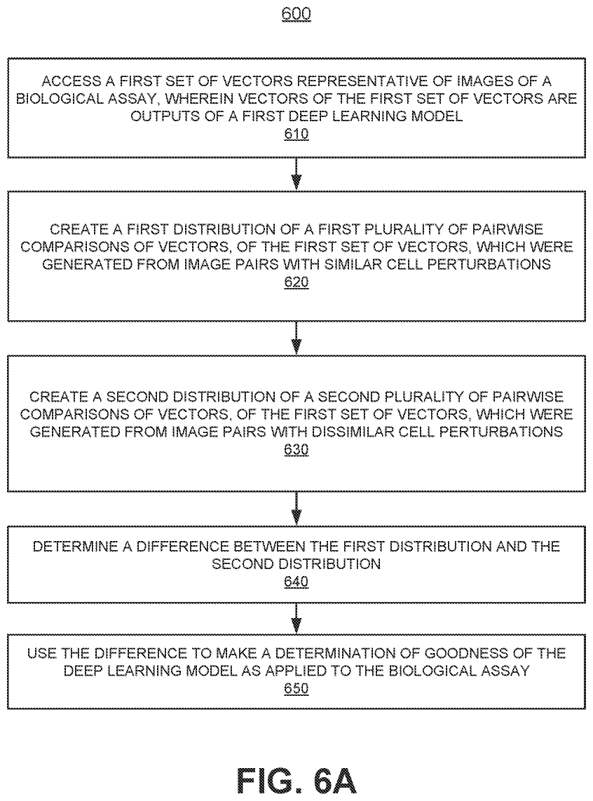

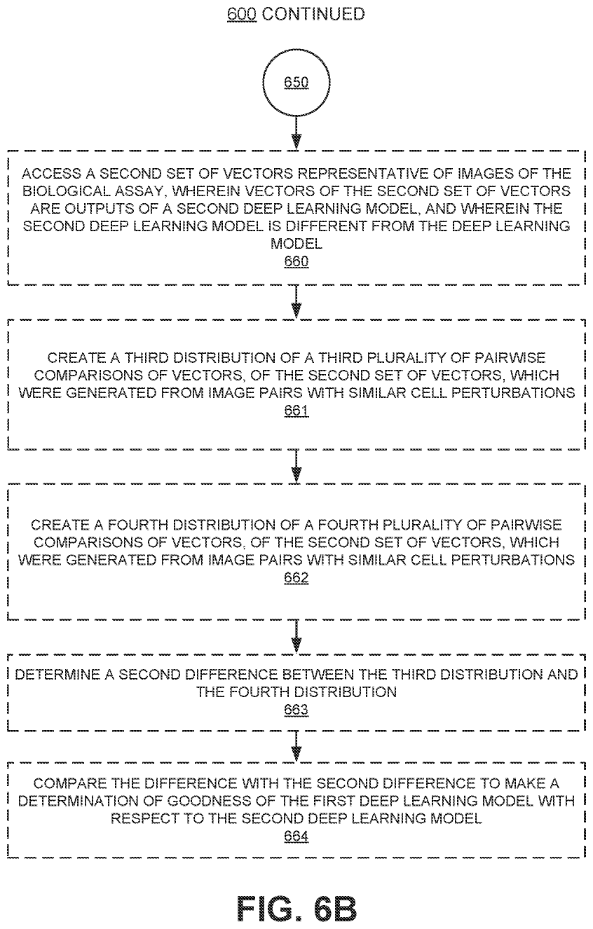

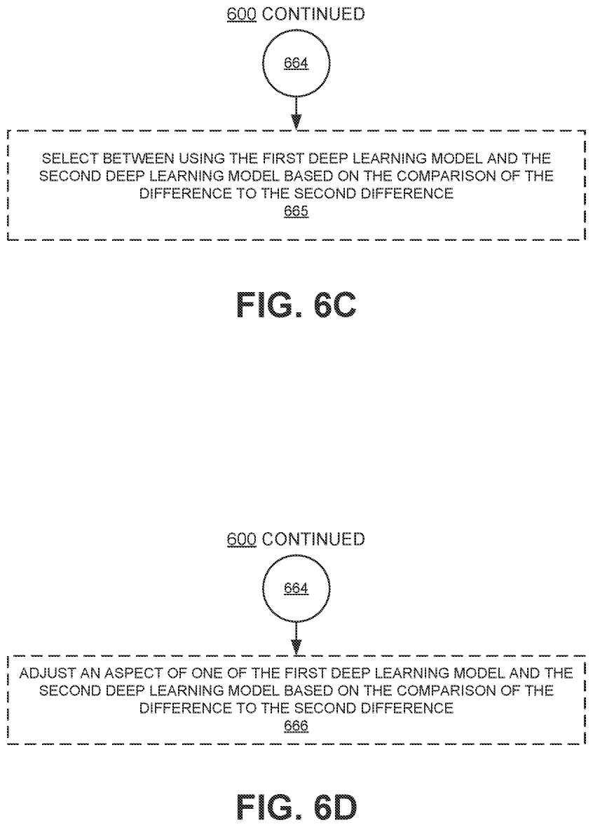

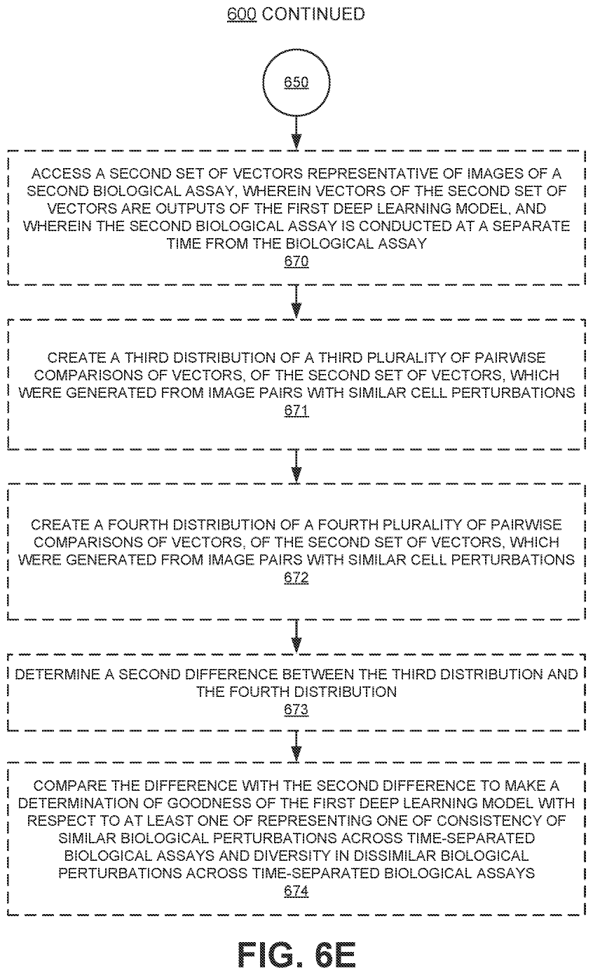

FIGS. 6A-6E illustrate a flow diagram of an example method of determining a goodness of a deep learning model, in accordance with various embodiments. Procedures of the methods illustrated by flow diagram 600 of FIGS. 6A-6E will be described with reference to aspects and/or components of one or more of FIGS. 1-5. It is appreciated that in some embodiments, the procedures may be performed in a different order than described in a flow diagram, that some of the described procedures may not be performed, and/or that one or more additional procedures to those described may be performed. Flow diagram 600 includes some procedures that, in various embodiments, are carried out by one or more processors or controllers (e.g., a processor 506, a computer system 110, or the like) under the control of computer-readable and computer-executable instructions that are stored on non-transitory computer readable storage media (e.g., peripheral computer readable storage media 502, ROM 510, RAM 508, data storage unit 512, or the like). It is further appreciated that one or more procedures described in flow diagram 600 may be implemented in hardware, or a combination of hardware with firmware and/or software.

With reference to FIG. 6A, at procedure 610 of flow diagram 600, in various embodiments, a first set of vectors representative of images of a biological assay is accessed. Vectors in the accessed first set of vectors are outputs of a first deep learning model. In some embodiments, this comprises computer system 110, or a processor 506 (e.g., 506A), accessing a store of vectors such as set of vectors 121-1 (see FIG. 1) which may be located in a database or other store internal or external to computer system 110.

With continued reference to FIG. 6A, at procedure 620 of flow diagram 600, in various embodiments, a first distribution is created of a first plurality of pairwise comparisons of vectors of the first set of vectors. The vectors used were generated from image pairs (of images of the biological assay) with similar cell perturbations. In some embodiments, this comprises computer system 110, or a processor 506, selecting pairs of vectors from vectors 121-1 which have similar biology (e.g., the same cell lines and the same perturbations in the images from which the vectors are generated) and then comparing the vectors for each of the similar pairs to determine the similarity in the compared vectors. Computer system 110, or a processor 506, then compiles the pairwise comparisons into a distribution (e.g., a cumulative distribution function). With reference to FIG. 2, distribution 210 is one example of a distribution. The comparisons may measure the similarity between compared pairs in distance apart (e.g., Euclidian distance between vectors), the angle between the compared vectors, the cosine of the angle between the compared vectors, or other technique for determining similarity of two vectors. Accordingly, the first distribution may be a distribution that represents the similarities as distances, angles, cosine comparisons, etc.

With continued reference to FIG. 6A, at procedure 630 of flow diagram 600, in various embodiments, a second distribution is created of a second plurality of pairwise comparisons of vectors of the first set of vectors. The vectors used were generated from image pairs (of images of the biological assay) with dissimilar cell perturbations. In some embodiments, this comprises computer system 110, or a processor 506, selecting second pairs of vectors from vectors 121-1 which have dissimilar biology (e.g., the same cell lines in each member of the pair, but different perturbations in each member of the pair, in the images from which the vectors are generated) and then comparing the vectors for each of the differing pairs to determine the similarity in the compared vectors. Computer system 110, or a processor 506, then compiles the second pairwise comparisons into a second distribution (e.g., a cumulative distribution function). With reference to FIG. 2, distribution 220 is one example of a second distribution. The comparisons performed are typically the same type of comparisons performed in the pairwise comparison described in procedure 620. Accordingly, the second distribution may be a distribution that represents the similarities of the second pairs (i.e., the same/different pairs) in the same manner as the similarities of the pairs (i.e., the same/same pairs) compared in procedure 620.

With continued reference to FIG. 6A, at procedure 640 of flow diagram 600, in various embodiments, a difference is determined between the first distribution and the second distribution. In some embodiments, this comprises computer system 110, or a processor 506, determining the difference. With reference to FIG. 2, difference 230 is one example of a difference metric between distribution 210 and second distribution 220. The difference is a metric that represents some aspect of the separation between the distribution of procedure 620 and the distribution of procedure 630. For example, it may be the maximum vertical separation, the minimum vertical separation, the average vertical separation, or some other measure of distance between the first distribution and the second distribution. In some embodiments, the difference may be determined by a parametric test that makes some assumptions about the distributions. In other embodiments, the difference may be determined by a non-parametric test which does not make any assumptions about the distributions. Some non-limiting examples of non-parametric tests include, but are not limited to: performing a K-S test; performing a Kolmogorov-Smirnov test to determine the difference metric between the distribution and the second distribution; or performing a Wilcoxon Rank-Sum test to determine the difference metric between the distribution and the second distribution.

With continued reference to FIG. 6A, at procedure 650 of flow diagram 600, in various embodiments, the difference is used to make a determination of goodness of the deep learning model as applied to the biological assay. In some embodiments, this comprises computer system 110, or a processor 506, accessing comparing the magnitude of the difference to a benchmark or threshold. For example, if a desired threshold is exceeded by the difference, then it is determined that the deep learning model used to transform the relevant biology of images into vectors 121-1 has a suitable level of goodness. As previously mentioned, the goodness measure is a simultaneous measure of consistency of the deep learning model in representing similar biology as similar in vectors 121-1 and the ability of the model to accurately preserve diversity of dissimilar biology in set of vectors 121-1. The goodness increases the more the difference exceeds the threshold. If the difference does not exceed the threshold, then the goodness of the model may be judged to be low or unsuitable. The greater the distance between the difference and reaching the threshold, the lower the goodness of the model. In this manner, assessing the relative goodness of a model and the vectors produced by the model, the goodness of a biological vector space containing the vectors can be determined and/or characterized.

With reference to FIG. 6B, at procedure 660 of flow diagram 600, in various embodiments, the method as described in 610-650, further includes accessing a second set of vectors representative of images of the biological assay. The second set of vectors is an output of a second deep learning model which is different from the first deep learning model. In some embodiments, this comprises computer system 110, or a processor 506 accessing a set of vectors, such as vectors 121-4 (see FIG. 1), which may be located in a database 120 or other store internal or external to computer system 110.

With continued reference to FIG. 6B, at procedure 661 of flow diagram 600, in various embodiments, a third distribution is created of a third plurality of pairwise comparisons of vectors of the second set of vectors. The vectors used were generated from image pairs (of images of the biological assay) with similar cell perturbations. In some embodiments, this comprises computer system 110, or a processor 506, selecting third pairs of vectors from vectors 121-4 which have similar biology (e.g., the same cell lines and the same perturbations) and then comparing the same vector for each of the third pairs to determine the similarity in the compared vectors. Computer system 110, or a processor 506, then compiles the pairwise comparisons into a third distribution (e.g., a cumulative distribution function). With reference to FIG. 3, distribution 310 is one example of a third distribution. The comparisons may measure the similarity between compared third pairs in distance apart (e.g., Euclidian distance between compared vectors), the angle between the compared vectors, the cosine of the angle between the compared vectors, or other technique for determining similarity of two vectors. Accordingly, the third distribution may be a distribution that represents the similarities as distances, angles, cosine comparisons, etc. In practice, the comparison used to determine similarity of the third pairs will be the same comparison used in procedure 620 of FIG. 6A, as this facilitates comparison of respective difference metrics associated with vectors 121-1 and vectors 121-2.

With continued reference to FIG. 6B, at procedure 662 of flow diagram 600, in various embodiments, a fourth distribution is created of a third plurality of pairwise comparisons of vectors of the second set of vectors. The vectors used were generated from image pairs (of images of the biological assay) with dissimilar cell perturbations. In some embodiments, this comprises computer system 110, or a processor 506, selecting fourth pairs of vectors from vectors 121-4 which have dissimilar biology (e.g., the same cell lines in each member of the pair, but different perturbations in each member of the pair) and then comparing the same vector for each of the fourth pairs to determine the similarity in the compared vectors. Computer system 110, or a processor 506, then compiles the fourth pairwise comparisons into a fourth distribution (e.g., a cumulative distribution function). With reference to FIG. 3, distribution 320 is one example of a fourth distribution. The comparisons performed are typically the same type of comparisons performed in the pairwise comparison described in procedure 620 of FIG. 6A. Accordingly, the fourth distribution may be a distribution that represents the similarities of the fourth pairs (i.e., the same/different pairs) in the same manner as the similarities of the pairs (i.e., the same/same pairs) compared in procedure 620.

With continued reference to FIG. 6B, at procedure 663 of flow diagram 600, in various embodiments, a second difference is determined between the third distribution and the fourth distribution. In some embodiments, this comprises computer system 110, or a processor 506, determining the second difference. With reference to FIG. 3, difference 330 is one example of a second difference metric between distribution 310 and second distribution 320. The second difference is a metric that represents some aspect of the separation between the distribution of procedure 661 and the distribution of procedure 662. For example, it may be the maximum vertical separation, the minimum vertical separation, the average vertical separation, or some other measure of distance between the first distribution and the second distribution. In some embodiments, the difference may be determined by a parametric test that makes some assumptions about the distributions. In other embodiments, the difference may be determined by a non-parametric test which does not make any assumptions about the distributions. Some non-limiting examples of non-parametric tests include, but are not limited to: performing a K-S test; performing a Kolmogorov-Smirnov test to determine the difference metric between the distribution and the second distribution; or performing a Wilcoxon Rank-Sum test to determine the difference metric between the distribution and the second distribution. In practice, the mechanism used to measure the second difference will be the same one used in procedure 640 to measure the first difference, as this facilitates comparison of respective difference metrics associated with vectors 121-1 and vectors 121-4.

With continued reference to FIG. 6B, at procedure 664 of flow diagram 600, in various embodiments, the difference is compared with the second difference to make a determination of goodness of the first deep learning model with respect to the second deep learning model. In some embodiments, this comprises computer system 110, or a processor 506, accessing comparing the magnitude of the difference to the magnitude of the second difference, with the larger of the two differences being adjudged to denote more an underlying deep learning model with more relative goodness than the smaller of the two differences.

With reference to FIG. 6C, at procedure 665 of flow diagram 600, in various embodiments, the method as described in procedures 610-664 further includes selecting between using the first deep learning model and the second deep learning model based on the comparison of the difference to the second difference. In some embodiments, this comprises computer system 110, or a processor 506, selecting between of first learning model and the second deep learning model based on which one is associated with the larger of the compared differences. In some instances, the selection may result in the selected deep learning model seeing additional use for the vectorization of the cell biology of images, and/or the non-selected deep learning model seeing diminished or ceased use in the vectorization of the cell biology of images.

With reference to FIG. 6D, at procedure 666 of flow diagram 600, in various embodiments, the method as described in procedures 610-664 further includes adjusting an aspect of one of the first deep learning model and the second deep learning model based on the comparison of the difference to the second difference. In some embodiments, this comprises computer system 110, or a processor 506, analyzing the two deep learning models to determine a difference in procedures, filters, or tools available to them for deep learning and then adding a procedure, filter, or tool from the selected deep learning model which is not present in the non-selected deep learning model. In some embodiments, this comprises computer system 110, or a processor 506, analyzing the two deep learning models to determine a difference in procedures, filters, or tools available to them for deep learning and then removing a procedure, filter, or tool from the non-selected deep learning model which is not present in the selected deep learning model. In some embodiments, other adjustments may be made such as adjusting an aspect of the non-selected deep learning model to match or more closely match a similar aspect (e.g., a filter weight) in the selected deep learning model.

With reference to FIG. 6E, at procedure 670 of flow diagram 600, in various embodiments, the method as described in 610-650, further includes accessing a second set of vectors representative of images of a second biological assay. The vectors of the second set of vectors are outputs of the first deep learning model. However, the second biological assay is conducted at a separate time (e.g., in a separate run or batch separated by hours, days, weeks, or longer) from the first biological assay. In some embodiments, this comprises computer system 110, or a processor 506 accessing vectors such as vectors 121-3 (see FIG. 1) which may be located in a database 120 or other store internal or external to computer system 110.

With continued reference to FIG. 6E, at procedure 671 of flow diagram 600, in various embodiments, a third distribution is created of a third plurality of pairwise comparisons of vectors of the second set of vectors. The vectors used were generated from image pairs (of images of the biological assay) with similar cell perturbations. In some embodiments, this comprises computer system 110, or a processor 506, selecting third pairs of vectors from vectors 121-3 which have similar biology (e.g., the same cell lines and the same perturbations) and then comparing the same vector for each of the third pairs to determine the similarity in the compared vectors. Computer system 110, or a processor 506, then compiles the pairwise comparisons into a third distribution (e.g., a cumulative distribution function). With reference to FIG. 4, distribution 410 is one example of a third distribution. The comparisons may measure the similarity between compared third pairs in distance apart (e.g., Euclidian distance between compared vectors), the angle between the compared vectors, the cosine of the angle between the compared vectors, or other technique for determining similarity of two vectors. Accordingly, the third distribution may be a distribution that represents the similarities as distances, angles, cosine comparisons, etc. In practice, the comparison used to determine similarity of the third pairs will be the same comparison used in procedure 620 of FIG. 6A, as this facilitates comparison of respective difference metrics associated with vectors 121-1 and vectors 121-3.

With continued reference to FIG. 6E, at procedure 672 of flow diagram 600, in various embodiments, a fourth distribution is created of a third plurality of pairwise comparisons of vectors of the second set of vectors. The vectors used were generated from image pairs (of images of the biological assay) with dissimilar cell perturbations. In some embodiments, this comprises computer system 110, or a processor 506, selecting fourth pairs of vectors from vectors 121-3 which have dissimilar biology (e.g., the same cell lines in each member of the pair, but different perturbations in each member of the pair) and then comparing the same vector for each of the fourth pairs to determine the similarity in the compared vectors. Computer system 110, or a processor 506, then compiles the fourth pairwise comparisons into a fourth distribution (e.g., a cumulative distribution function). With reference to FIG. 4, distribution 420 is one example of a fourth distribution. The comparisons performed are typically the same type of comparisons performed in the pairwise comparison described in procedure 620. Accordingly, the fourth distribution may be a distribution that represents the similarities of the fourth pairs (i.e., the same/different pairs) in the same manner as the similarities of the pairs (i.e., the same/same pairs) compared in procedure 620.

With continued reference to FIG. 6E, at procedure 673 of flow diagram 600, in various embodiments, a second difference is determined between the third distribution and the fourth distribution. In some embodiments, this comprises computer system 110, or a processor 506, determining the second difference. With reference to FIG. 4, difference 430 is one example of a second difference metric between distribution 410 and second distribution 420. The second difference is a metric that represents some aspect of the separation between the distribution of procedure 671 and the distribution of procedure 672. For example, it may be the maximum vertical separation, the minimum vertical separation, the average vertical separation, or some other measure of distance between the first distribution and the second distribution. In some embodiments, the difference may be determined by a parametric test that makes some assumptions about the distributions. In other embodiments, the difference may be determined by a non-parametric test which does not make any assumptions about the distributions. Some non-limiting examples of non-parametric tests include, but are not limited to: performing a K-S test; performing a Kolmogorov-Smirnov test to determine the difference metric between the distribution and the second distribution; or performing ag a Wilcoxon Rank-Sum test to determine the difference metric between the distribution and the second distribution. In practice, the mechanism used to measure the second difference will be the same one used in procedure 640 to measure the first difference, as this facilitates comparison of respective difference metrics associated with vectors 121-1 and vectors 121-3.

With continued reference to FIG. 6E, at procedure 674 of flow diagram 600, in various embodiments, comparing the difference with the second difference to make a determination of goodness of the first deep learning model with respect to at least one of representing one of consistency of similar biological perturbations across time-separated biological assays and diversity in dissimilar biological perturbations across time-separated biological assays. In some embodiments, this comprises computer system 110, or a processor 506, accessing comparing the magnitude of the difference to the magnitude of the second difference, with the larger of the two differences. Consider a first case where the difference and second difference are the same or substantially the same (e.g., withing some prespecified margin such as 5% of the value of one another), then the first learning model may be deemed to maintain both consistency and diversity across time-separated biological assays. In such a case, this may facilitate combining of datasets of the time-separated first and second biological assays. Consider a second case wherein the first and second differences differ beyond some prespecified margin (e.g., differ by greater than 5% in value, or 10% in value, or other prespecified margin), then the first deep learning model may be deemed not to maintain either or both of consistency and diversity across time-separated biological assays. In such a case, this may preclude combining of datasets of the time-separated first and second biological assays.

### Conclusion

The examples set forth herein were presented in order to best explain, to describe particular applications, and to thereby enable those skilled in the art to make and use embodiments of the described examples. However, those skilled in the art will recognize that the foregoing description and examples have been presented for the purposes of illustration and example only. The description as set forth is not intended to be exhaustive or to limit the embodiments to the precise form disclosed. Rather, the specific features and acts described above are disclosed as example forms of implementing the claims.

Reference throughout this document to “one embodiment,” “certain embodiments,” “an embodiment,” “various embodiments,” “some embodiments,” or similar term means that a particular feature, structure, or characteristic described in connection with the embodiment is included in at least one embodiment. Thus, the appearances of such phrases in various places throughout this specification are not necessarily all referring to the same embodiment. Furthermore, the particular aspects, features, structures, or characteristics of any embodiment may be combined in any suitable manner with one or more other aspects, features, structures, or characteristics of one or more other embodiments without limitation.

What is claimed is:

1. A system for determining a goodness of a deep learning model, comprising:
a memory; and
at least one processor coupled with the memory and configured to:
access a first set of vectors representative of images of a biological assay, wherein vectors of the first set of vectors are outputs of a first deep learning model;
create a first distribution of a first plurality of pairwise comparisons of vectors, of the first set of vectors, which were generated from image pairs with similar cell perturbations;
create a second distribution of a second plurality of pairwise comparisons of vectors, of the first set of vectors, which were generated from image pairs with dissimilar cell perturbations;
determine a difference between the first distribution and the second distribution; and
use the difference to make a determination of goodness of the first deep learning model as applied to the biological assay.

2. The system of claim 1, wherein the processor is further configured to:
access a second set of vectors representative of images of the biological assay, wherein vectors of the second set of vectors are outputs of a second deep learning model, and wherein the second deep learning model is different from the deep learning model;
create a third distribution of a third plurality of pairwise comparisons of vectors, of the second set of vectors, which were generated from image pairs with similar cell perturbations;
create a fourth distribution of a fourth plurality of pairwise comparisons of vectors, of the second set of vectors, which were generated from image pairs with similar cell perturbations;
determine a second difference between the third distribution and the fourth distribution; and
compare the difference with the second difference to make a determination of goodness of the first deep learning model with respect to the second deep learning model.

3. The system as recited in claim 2, wherein the processor is further configured to:
select between using the first deep learning model and the second deep learning model based on the comparison of the difference to the second difference.

4. The system as recited in claim 2, wherein the processor is further configured to:
adjust an aspect of one of the first deep learning model and the second deep learning model based on the comparison of the difference to the second difference.

5. The system of claim 1, wherein the processor is further configured to:
access a second set of vectors representative of images of a second biological assay, wherein vectors of the second set of vectors are outputs of the first deep learning model, and wherein the second biological assay is conducted at a separate time from the biological assay;
create a third distribution of a third plurality of pairwise comparisons of vectors, of the second set of vectors, which were generated from image pairs with similar cell perturbations;
create a fourth distribution of a fourth plurality of pairwise comparisons of vectors, of the second set of vectors, which were generated from image pairs with similar cell perturbations;
determine a second difference between the third distribution and the fourth distribution; and
compare the difference with the second difference to make a determination of goodness of the first deep learning model with respect to at least one of representing consistency of similar biological perturbations across time-separated biological assays and representing diversity in dissimilar biological perturbations across time-separated biological assays.

6. The system of claim 1, wherein the processor configured to create a first distribution comprises the processor being configured to:
create the first distribution to represent the first plurality of pairwise comparisons of vectors as one of distances and angle comparisons.

7. The system of claim 1, wherein the processor configured to create a first distribution comprises the processor being configured to:
perform one of a parametric test and a non-parametric test.

8. A method of determining a goodness of a deep learning model, comprising:
accessing a first set of vectors representative of images of a biological assay, wherein vectors of the first set of vectors are outputs of a first deep learning model;
creating a first distribution of a first plurality of pairwise comparisons of vectors, of the first set of vectors, which were generated from image pairs with similar cell perturbations;
creating a second distribution of a second plurality of pairwise comparisons of vectors, of the first set of vectors, which were generated from image pairs with dissimilar cell perturbations;
determining a difference between the first distribution and the second distribution; and
using the difference to make a determination of goodness of the first deep learning model as applied to the biological assay.

9. The method as recited in claim 8, further comprising:
accessing a second set of vectors representative of images of the biological assay, wherein vectors of the second set of vectors are outputs of a second deep learning model, and wherein the second deep learning model is different from the deep learning model;
creating a third distribution of a third plurality of pairwise comparisons of vectors, of the second set of vectors, which were generated from image pairs with similar cell perturbations;
creating a fourth distribution of a fourth plurality of pairwise comparisons of vectors, of the second set of vectors, which were generated from image pairs with similar cell perturbations;
determining a second difference between the third distribution and the fourth distribution; and
comparing the difference with the second difference to make a determination of goodness of the first deep learning model with respect to the second deep learning model.

10. The method as recited in claim 9, further comprising:
selecting between using the first deep learning model and the second deep learning model based on the comparison of the difference to the second difference.

11. The method as recited in claim 9, further comprising:
adjusting an aspect of one of the first deep learning model and the second deep learning model based on the comparison of the difference to the second difference.

12. The method as recited in claim 8, further comprising:
accessing a second set of vectors representative of images of a second biological assay, wherein vectors of the second set of vectors are outputs of the first deep learning model, and wherein the second biological assay is conducted at a separate time from the biological assay;
creating a third distribution of a third plurality of pairwise comparisons of vectors, of the second set of vectors, which were generated from image pairs with similar cell perturbations;
creating a fourth distribution of a fourth plurality of pairwise comparisons of vectors, of the second set of vectors, which were generated from image pairs with similar cell perturbations;
determining a second difference between the third distribution and the fourth distribution; and
comparing the difference with the second difference to make a determination of goodness of the first deep learning model with respect to at least one of representing consistency of similar biological perturbations across time-separated biological assays and representing diversity in dissimilar biological perturbations across time-separated biological assays.

13. The method as recited in claim 8, wherein the creating a first distribution of a first plurality of pairwise comparisons of vectors, of the first set of vectors, which were generated from image pairs with similar cell perturbations comprises:
creating the first distribution to represent the first plurality of pairwise comparisons of vectors as distances.

14. The method as recited in claim 8, wherein the creating a first distribution of a first plurality of pairwise comparisons of vectors, of the first set of vectors, which were generated from image pairs with similar cell perturbations comprises:
creating the first distribution to represent the first plurality of pairwise comparisons of vectors as angles.

15. The method as recited in claim 8, wherein the determining a difference between the first distribution and the second distribution comprises:
performing one of a parametric test and a non-parametric test.

16. The method as recited in claim 8, wherein the determining a difference between the first distribution and the second distribution comprises:
performing a Kolmogorov-Smirnov test.

17. The method as recited in claim 8, wherein the determining a difference between the first distribution and the second distribution comprises:
performing a Wilcoxon Rank-Sum test.

18. The method as recited in claim 8, wherein the determining a difference between the first distribution and the second distribution comprises:
performing a Kolmogorov-Shapiro test.

19. The method as recited in claim 8, wherein determining a difference between the first distribution and the second distribution comprises:
calculating a measure of distance between the first distribution and the second distribution.

20. A non-transitory computer readable storage medium comprising instructions embodied thereon, which when executed, cause a processor to perform a method of determining a goodness of a deep learning model, comprising:
accessing a first set of vectors representative of images of a biological assay, wherein vectors of the first set of vectors are outputs of a first deep learning model;
creating a first distribution of a first plurality of pairwise comparisons of vectors, of the first set of vectors, which were generated from image pairs with similar cell perturbations;
creating a second distribution of a second plurality of pairwise comparisons of vectors, of the first set of vectors, which were generated from image pairs with dissimilar cell perturbations;
determining a difference between the first distribution and the second distribution; and
using the difference to make a determination of goodness of the first deep learning model as applied to the biological assay.

21. The non-transitory computer readable storage medium of claim 20, wherein the method further comprises:
accessing a second set of vectors representative of images of the biological assay, wherein vectors of the second set of vectors are outputs of a second deep learning model, and wherein the second deep learning model is different from the deep learning model;
creating a third distribution of a third plurality of pairwise comparisons of vectors, of the second set of vectors, which were generated from image pairs with similar cell perturbations;
creating a fourth distribution of a fourth plurality of pairwise comparisons of vectors, of the second set of vectors, which were generated from image pairs with similar cell perturbations;
determining a second difference between the third distribution and the fourth distribution; and
comparing the difference with the second difference to make a determination of goodness of the first deep learning model with respect to the second deep learning model.

22. The non-transitory computer readable storage medium of claim 21, wherein the method further comprises:
selecting between using the first deep learning model and the second deep learning model based on the comparison of the difference to the second difference.

23. The non-transitory computer readable storage medium of claim 21, wherein the method further comprises:
adjusting an aspect of one of the first deep learning model and the second deep learning model based on the comparison of the difference to the second difference.

24. The non-transitory computer readable storage medium of claim 20, wherein the method further comprises:
accessing a second set of vectors representative of images of a second biological assay, wherein vectors of the second set of vectors are outputs of the first deep learning model, and wherein the second biological assay is conducted at a separate time from the biological assay;
creating a third distribution of a third plurality of pairwise comparisons of vectors, of the second set of vectors, which were generated from image pairs with similar cell perturbations;
creating a fourth distribution of a fourth plurality of pairwise comparisons of vectors, of the second set of vectors, which were generated from image pairs with similar cell perturbations;
determining a second difference between the third distribution and the fourth distribution; and
comparing the difference with the second difference to make a determination of goodness of the first deep learning model with respect to at least one of representing consistency of similar biological perturbations across time-separated biological assays and representing diversity in dissimilar biological perturbations across time-separated biological assays.
